(19) United States
(12) Patent Application Publication
(10) Pub. No.: US 2006/0127333 A1
(43) Pub. Date: Jun. 15, 2006

(54) Cosmetic compositions containing very high viscosity silicone gum

(76) Inventors: Anjali Abhimanyu Patil, Westfield, NJ (US); Robert Walter Sandewicz, Monroe Township, NJ (US); Joseph Frank Calello, Bridgewater, NJ (US); Tatyana Zamyatin, Princeton Junction, NJ (US); Ida Marie Sandewicz, Monroe Township, NJ (US); Jean Marie Manelski, Spring Lake, NJ (US); Jodi Lorraine Kochman, Point Pleasant Beach, NJ (US)

Correspondence Address: Julie Blackburn;Revlon Consumer Products Corporation, 237 Park Avenue, New York, NY, 10017, US

(21) Appl. No.: 11/010,725

(22) Filed: Dec. 13, 2004

(51) Int. Cl. A61K 8/89

(52) U.S. Cl. 424/63

(57) ABSTRACT

A color cosmetic composition comprising at least one very high viscosity silicone gum in a cosmetically acceptable carrier.

### TECHNICAL FIELD

The invention is in the field of cosmetic compositions for application to keratinous surfaces for the purposes of conditioning, coloring, and otherwise beautifying the keratinous surface.

### BACKGROUND OF THE INVENTION

The price of entry in the color cosmetic category is long wear. Consumers are no longer satisfied with color cosmetics that do not wear for at least a few hours before reapplication is necessary. There are a variety of ways to achieve long wear in cosmetic compositions. One way is through the use of polymers that have an affinity to keratinous surfaces. Examples of such polymers include silicone resins or synthetic organic polymers. When applied to the keratinous surface in the form of a color cosmetic composition, the polymers facilitate adhesion of the composition to the surface resulting in improved wear.

Various types of pressure sensitive adhesives (PSA's) have been touted for use in long wearing products. The functional characteristics of PSA's (affinitive, adherent) are exactly the properties that contribute to long wear. Silicone gums are well known PSA's. Such PSA's are often used in industrial applications, most notably in affixing labels to cartons, or cementing other types of surfaces together. Lower viscosity silicone gums (less than 10 million centistokes at 25° C.) are also known for use in cosmetic compositions. Such silicone gums are of a low enough viscosity to permit ready formulation into the cosmetic product being made. Silicone gums having very high viscosities are not known for use in cosmetics because they are very difficult to handle and generally not commercially available. It has been discovered that silicone gums having very high viscosity (greater than about 12 million centistokes at 25° C.) provide wear and adhesion to keratinous surfaces that is improved when compared to lower viscosity silicone gums. In particular, silicone gums having viscosities ranging from 12 million to 900 million are particularly useful in providing cosmetic compositions that have long wear or transfer resistance and adhesion to keratinous surfaces.

Accordingly, it is an object of the invention to provide color cosmetic compositions comprising at least one very high viscosity silicone gum.

It is a further object of the invention to provide cosmetic compositions comprising silicone gums having a viscosity ranging from about 12 million to 900 million.

It is a further object of the invention to provide cosmetic compositions comprising a very high viscosity silicone gum solvated in at least one viscosity decreasing composition compatible solvent.

It is a further object of the invention to provide color cosmetic compositions, in one specific embodiment, containing at least one silicone gum having a viscosity ranging from about 12 million to 900 million wherein the composition contains reduced amounts, or is free of, crystalline structuring agents such as waxes.

### SUMMARY OF THE INVENTION

The invention is directed to a color cosmetic composition comprising at least one very high viscosity silicone gum in a cosmetically acceptable carrier.

The invention is directed to a cosmetic composition containing at least one silicone gum having a viscosity ranging from about 12 million to about 900 million centistokes at 25° C.

The invention is directed to a cosmetic composition comprising a very high viscosity silicone gum solvated in at least one viscosity decreasing, composition compatible solvent.

The invention is directed to a cosmetic composition containing at least one silicone gum having a viscosity ranging from about 12 million to about 900 million centistokes at 25° C. wherein the composition contains reduced amounts, or is free of, crystalline structuring agents.

### DETAILED DESCRIPTION

All percentages mentioned herein are percentages by weight unless otherwise indicated. Further, any documents mentioned herein are incorporated by reference in their entirety.

I. The Compositions of the Invention

A. Silicone Gum

The compositions of the invention comprise at least one very high viscosity silicone gum. The term “very high viscosity” with respect to the silicone gum means a silicone polymer that has a sufficient degree of polymerization to form a gum having a viscosity ranging from about 12 million to about 900 million centistokes at 25° C., preferably from about 15 million to about 750 million, more preferably from about 20 million to about 600 million centistokes, even more preferably from about 20 million to 500 million centistokes. All ranges mentioned herein include all subranges, e.g. 20, 21, 22, 23, 24, 25, 26, million, etc. The silicone gum may be in the crosslinked or uncrosslinked form.

The silicone gums that are used in the compositions include, but are not limited to, those of the general formula: 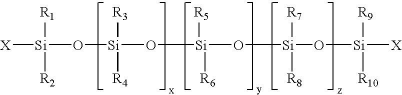
wherein:

R₁ through R₉ are each independently an alkyl having 1 to 30 carbon atoms, aryl, or aralkyl; and X is OH or a C₁₋₃₀ alkyl, hydroxyl, or vinyl; and wherein x, y, or z may be zero provided no more than two of x, y, or z are zero at any one time, and further that x, y, and z are such that the silicone gum has a viscosity of at least about 12 million centistokes, ranging up to about 900 million centistokes at 25° C. Preferred is where R is X or OH.

Such silicone gums may be purchased from a variety of silicone manufacturers including Wacker-Chemie or Dow Corning, and the like. Silicone gums having the viscosity ranges mentioned above may be purchased from Wacker-Chemie or Dow Corning Corporation. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Corning Corporation under the trade name 1401. It is further noted that in one embodiment of the invention the silicone gum is not dimethiconol. In another embodiment of the invention the silicone gum is dimethiconol, and is found in combination with at least one linear volatile silicone.

B. Other Ingredients

The compositions of the invention may comprise a variety of other ingredients, including but not limited to those set forth below. Additionally, the compositions of the invention may be in the form of lipsticks, eyeshadows, blushes, concealers, mascara, eyeliner, foundation makeup, and the like. The compositions may be in the anhydrous or emulsion form.

1. Volatile Solvents

The composition may comprise one or more volatile solvents. In the event the inventive compositions are transfer resistant or even long wearing, it is desirable that they contain at least one volatile solvent. If present, suggested ranges are from about 0.1-95%, preferably about 0.5-90%, more preferably about 1-80% by weight of the total composition. Suitable volatile solvents may be silicones or paraffinic hydrocarbons. The term “volatile” when used herein, means that the solvent has a vapor pressure of at least about 2 mm. of mercury at 20° C.

(a). Volatile Silicones

Suitable volatile silicones include linear or cyclic volatile silicones. Such volatile silicones generally have a viscosity ranging from about 0.1 to 10, preferably about 0.1-5 centistokes at 25° C.

Cyclic silicones (or cyclomethicones) are of the general formula: 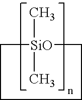
where n=3-6.

Linear volatile silicones in accordance with the invention have the general formula:
(CH₃)₃Si—O—[Si(CH₃)₂—O]ₙ—Si(CH₃)₃
where n=0, 1, 2, 3, 4, 5, 6, or 7, preferably 0, 1, 2, 3, 4, or 5, more preferably 1, 2, 3, or 4.

Linear and cyclic volatile silicones are available from various commercial sources including Dow Corning Corporation and General Electric. The Dow Corning volatile silicones are sold under the tradenames Dow Corning 244, 245, 344, and 200 fluids. Suitable linear volatile silicones include hexamethyldisiloxane, octamethyltrisiloxane, decamethyltetrasiloxane, dodecamethylpentasiloxane, and the like. Suitable cyclic volatile silicones include octamethylcyclotetrasiloxane, decamethylcyclopentasiloxane, dodecamethylcyclohexasiloxane, and the like.

(b). Volatile Paraffinic Hydrocarbons

Also suitable are various straight or branched chain paraffinic hydrocarbons, for example, those having from 5 to 20 carbon atoms, more preferably 8-19 carbon atoms. Suitable hydrocarbons include pentane, hexane, heptane, decane, dodecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight ranging from about 70 to 225, preferably about 160 to 190, and a boiling point range of about 30 to 320, preferably about 60 to 260° C., and a viscosity of less than about 10 centistokes at 25° C. Such paraffinic hydrocarbons are available from EXXON under the ISOPARS trademark, and from the Permethyl Corporation. Suitable C₁₂ isoparaffins having the INCI name isododecane are manufactured by Permethyl Corporation under the tradename Permethyl 99A. Various C₁₆ isoparaffins commercially available under the trade name Permethyl R and having the INCI name isohexadecane are also suitable. Another suitable type of suitable paraffin is referred to as C9-11 isoparaffins, which is a mixture of isoparaffinic hydrocarbons having 9, 10, and 11 carbon atoms or C8-9 isoparaffins, which are a mixture of isoparaffinic hydrocarbons having 8 and 9 carbon atoms.

2. Structuring Agents

If desired, the composition may contain one or more structuring agents which will provide viscosity or thickness to the composition. If present, suggested ranges of structuring agents are from about 0.01-85%, preferably about 0.5-80%, more preferably about 1-75% by weight of the total composition. Suitable structuring agents include waxes, montmorillonite minerals, associative thickeners, and the like. Structuring agents may be crystalline or non-crystalline. Examples of crystalline structuring agents include waxes which exhibit a crystalline structure. Examples of non-crystalline structuring agents include montmorillonite minerals, polyamides, silicone polyamides, and other wax-like materials that are optically clear. It is further noted that waxes or structuring materials that are opaque tend to be crystalline in nature.

Suitable waxes include animal, vegetable, mineral, and synthetic waxes, or silicone waxes. Generally such waxes have a melting point ranging from about 28 to 125° C., preferably about 30 to 100° C. Examples of waxes include acacia, beeswax, ceresin, synthetic wax, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG 6-20 beeswax, or PEG-12 carnauba wax.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on.

Suitable waxes are also set forth in U.S. Pat. No. 5,725,845 which is hereby incorporated by reference in its entirety.

Suitable montmorillonite minerals include natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, and bentones, either alone or in combination with carbonate activators such as propylene carbonate.

Other types of thickening agents include fatty acids or alcohols, optionally substituted with hydroxyl groups, for example 12-hydroxystearic acid. Such fatty acids or alcohols have the general formula R—COOH or R—OH respectively, where R is a straight or branched chain, saturated or unsaturated alkyl having from about 6 to 45 carbon atoms, wherein one or more of the alkyl groups may be substituted with functional groups such as hydroxy, alkoxy, alkyl, and so on.

Also suitable are various other types of polymeric structuring agents such as polyamides, silicone polyamides, and the like.

Preferred is where the structuring agents, if present, comprise synthetic waxes such as polyethylene, or montmorillonite minerals or derivatives thereof. In another preferred embodiment of the invention the composition contains a reduced amount of crystalline structuring agents (e.g. from about 0.01-3%, preferably about 0.05-2% by weight of the total composition), or is substantially free of crystalline structuring agents. By “substantially free” means that such crystalline structuring agents are not intentionally added to the composition.

3. Non-Volatile Organic Oils

The composition may contain one or more non-volatile oils. The term “non-volatile” when used herein means that the oil has a measurable vapor pressure, e.g. a vapor pressure of less than about 2 mm. of mercury at 20° C. The term “oil” when used herein means an ingredient that is a pourable liquid at room temperature. Such oils tend to be generally nonpolar, but may contain substituents or moieties that are polar in character. If present, the oils that may be used in the compositions of the invention include silicone oils, organic oils, or mixtures thereof. Such oils, if present, may range from about 0.1-98%, preferably 0.5-90%, more preferably about 1-80% by weight of the total composition and include, but are not limited to, those set forth herein.

(a). Hydrocarbon Oils

Suitable oils are various non-volatile hydrocarbon oils including isoparaffins and olefins, or polymeric alpha olefins or polyalphaolefins, having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, hydrogenated polydecene, polybutene, mineral oil, pentahydrosqualene, squalene, squalane, and mixtures thereof. Preferably such hydrocarbons have from greater than 20 to about 80 carbon atoms.

(b). Esters

Also suitable are various esters that may be in the form of mono-, di-, or triesters. Preferably, such esters have a viscosity ranging from about 5 to 1,000,000 centistokes at 25° C.

(i). Monoesters

Monoesters are generally formed by the reaction of a monocarboxylic acid having the formula R—COOH, wherein R is a straight or branched chain saturated or unsaturated alkyl having from 1 to 30 carbon atoms, or phenyl; and an alcohol having the formula R—OH wherein R is a straight or branched chain saturated or unsaturated alkyl having from about 1-30 carbon atoms, or phenyl. Both the alcohol and the acid may be substituted with one or more hydroxyl groups, and the carboxylic acid may be an alpha hydroxy acid. Either one or both of the acid or alcohol may be a “fatty” acid or alcohol, for example, may have from about 6 to 22 carbon atoms. Examples of monoester oils that may be used in the compositions of the invention include, but are not limited to, hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, isostearyl isononanoate, isononyl isononanoate, cetyl isononanoate, cetyl stearate, stearyl lactate, stearyl octanoate, octyl isononanoate, stearyl heptanoate, stearyl stearate, and so on.

(ii). Diesters

Suitable diesters that may be used in the compositions of the invention are the reaction product of a dicarboxylic acid and an aliphatic or aromatic alcohol, or a monocarboxylic acid and an aliphatic or aromatic alcohol containing at least two hydroxyl groups. The dicarboxylic acid may contain from 1 to 30 carbon atoms, and may be in the straight or branched chain, saturated or unsaturated form. The dicarboxylic acid may be substituted with one or more hydroxyl groups. The aliphatic or aromatic alcohol may also contain 1 to 30 carbon atoms, and may be in the straight or branched chain, saturated, or unsaturated form. The aliphatic or aromatic alcohol may be substituted with one or more substituents such as hydroxyl. Preferably, one or more of the acid or alcohol is a fatty acid or alcohol, i.e. contains 6-22 carbon atoms. The dicarboxylic acid may also be an alpha hydroxy acid. Examples of diester oils that may be used in the compositions of the invention include diisostearyl malate, dibutyl adipate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, isononyl isononanaote, isohexadecyl stearate. Also suitable are ingredients that are the reaction product of polyethylene glycol and one or more fatty acids such as PEG-4 diheptanoate, PEG-4 dioctanoate, and so on; or derivatives of glycerin and one or more fatty acids such as diglyceryl diiosostearate, diglyceryl distearate, and the like.

(iii). Triesters

Suitable triesters comprise the reaction product of a tricarboxylic acid and an aliphatic or aromatic alcohol, or the reaction of an aliphatic or aromatic alcohol having three or more hydroxyl groups with mono- or dicarboxylic acids. As with the mono- and diesters mentioned above, the acid and alcohol contain from 1 to 30 carbon atoms, and may be saturated or unsaturated, straight or branched chain, and may be substituted with one or more hydroxyl groups. Preferably, one or more of the acid or alcohol is a fatty acid or alcohol containing 6 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, or derivatives of glycerin and one or more fatty acids such as glyceryl trioctanoate, glyceryl triisostearate, and the like.

(iv). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(v). Glyceryl Esters of Fatty Acids

Also suitable for use as the oil are various naturally occuring glyceryl esters of fatty acids, or triglycerides. Both vegetable, animal, or synthetic sources may be used. Examples of such oils include castor oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

4. Non-Volatile Silicone Oils

The compositions of the invention may comprise one or more non-volatile silicone oils. If present, suggested ranges are from about 0.1 to 85%, preferably from about 1-80%, more preferably from about 2-75% by weight of the total composition. Suitable non-volatile silicones include dimethicones having a viscosity ranging from about 10 to 500,000 centistokes at 25° C., phenyl substituted silicones such as phenyl dimethicone, phenyl trimethicone, diphenyl dimethicone, and the like.

5. Particulates

When the compositions of the invention are colored or opaque they may contain amounts of particulates ranging from about 0.01-95%, more preferably about 0.5-18% of particulate matter having a particle size of 0.01 to 200, preferably 0.25-100 microns. The particulate matter may be colored or non-colored (for example white) non-pigmentitious powders that may give the composition an opaque or semi-opaque quality. Suitable non-pigmentitious powders include bismuth oxychloride, titanated mica, fumed silica, spherical silica, polymethylmethacrylate, micronized teflon, boron nitride, acrylate copolymers, aluminum silicate, aluminum starch octenylsuccinate, calcium silicate, cellulose, chalk, corn starch, glyceryl starch, hydrated silica, kaolin, maltodextrin, microcrystalline cellulose, rice starch, silica, talc, mica, titanium dioxide, zinc laurate, zinc myristate, zinc rosinate, alumina, calcium carbonate, dextran, nylon, silica silylate, silk powder, sericite, soy flour, tin oxide, titanium hydroxide, trimagnesium phosphate, walnut shell powder, or mixtures thereof. While titanium dioxide is commonly considered to be a white pigment when used in paints, in cosmetics it is used more for its ability to mute color, and/or provide an opaque or semi-opaque finish, then as a colorizing ingredient. The above mentioned powders may be surface treated with lecithin, amino acids, mineral oil, silicone, or various other agents either alone or in combination, which coat the powder surface and render the particles more lipophilic in nature. In some cases the particulates may be in the form of fibers, which have a cross-sectional shape and some degree of length which may range from 0.1 mm. or greater. The particulates may also be in the lamellar, spherical, or other forms. Examples of particulates in lamellar form include mica and similar types of particulates that are found in sheet or platelet form. Examples of particulates in spherical form including spherical silica, and the like. Examples of such fibers include silk, nylon, cellulose, rayon, teflon, and other types of synthetic or natural materials.

The particulate matter component also may comprise various organic and/or inorganic pigments, either alone or in combination with one or more non-pigmentatious powders.

The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

The composition may contain a mixture of both pigmentatious and non-pigmentatious particulate matter. The percentage of pigment used in the particulate matter component will depend on the type of cosmetic being formulated. Preferred is where the particulate phase comprises a mixture of pigmentatious and non-pigmentatious particulate matter, generally ranging from about 0.1-80% pigmentatious particulate matter to about 0.1-90% non-pigmentatious particulate.

5. Film Forming Polymers

The composition may comprise one or more film forming polymers in addition to the high viscosity silicone gum. Such polymers may be silicones or polymers with repeating organic moieties. If present, such film forming polymers may be found in ranges of about 0.001-50%, preferably about 0.01-45%, more preferably about 0.1-20% by weight of the total composition. Such film forming polymers may be present in the form of dispersed or solvated particles in water, or in other non-aqueous solvents such as paraffinic hydrocarbons, silicone oils, or organic oils. Examples of such film forming polymers include, but are not limited to, those set forth below.

(a). Copolymers of Silicone and Ethylenically Unsaturated Monomers

One type of film forming polymer that may be used in the compositions of the invention is obtained by reacting silicone moieties with ethylenically unsaturated monomers. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units.

One type of polymer that may be used as the film forming polymer is a vinyl-silicone graft or block copolymer having the formula: 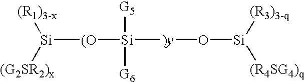
wherein G₅ represents monovalent moieties which can independently be the same or different selected from the group consisting of alkyl, aryl, aralkyl, alkoxy, alkylamino, fluoroalkyl, hydrogen, and -ZSA; A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z is methylene or propylene.

G₆ is a monovalent moiety which can independently be the same or different selected from the group consisting of alkyl, aryl, aralkyl, alkoxy, alkylamino, fluoroalkyl, hydrogen, and -ZSA;

G₂ comprises A;

G₄ comprises A;

R₁ is a monovalent moiety which can independently be the same or different and is selected from the group consisting of alkyl, aryl, aralkyl, alkoxy, alkylamino, fluoroalkyl, hydrogen, and hydroxyl; but preferably C₁₋₄ alkyl or hydroxyl, and most preferably methyl.

R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene, and

R₃ is a monovalent moiety that is independently alkyl, aryl, aralkyl, alkoxy, alkylamino, fluoroalkyl, hydrogen, or hydroxyl, preferably C₁₋₄ alkyl or hydroxyl, most preferably methyl;

R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene.

x is an integer of 0-3;

y is an integer of 5 or greater; preferably 10 to 270, and more preferably 40-270; and

q is an integer of 0-3.

These polymers are described in U.S. Pat. No. 5,468,477, which is hereby incorporated by reference in its entirety. One type of such a polymer is poly(dimethylsiloxane)-g-poly(isobutyl methacrylate), which is manufactured by 3-M Company under the tradename VS 70 IBM. This polymer may be purchased in the dry particulate form, or as a solution where the polymer is dissolved or dispersed in one or more of the liquids that may be found in the composition such as volatile oils (isododecane), water, or other non-volatile or volatile oils. When the polymer is found in the dry particulate form it can be dissolved in one or more of the liquids comprising the liquid carrier. This polymer has the CTFA name Polysilicone-6.

Another type of such a polymer comprises a vinyl, methacrylic, or acrylic backbone with pendant siloxane groups and pendant fluorochemical groups. Such polymers preferably comprise comprise repeating A, C, D and optionally B monomers wherein:

A is at least one free radically polymerizable acrylic or methacrylic ester of a 1,1,-dihydroperfluoroalkanol or analog thereof, omega-hydridofluoroalkanols, fluoroalkylsulfonamido alcohols, cyclic fluoroalkyl alcohols, and fluoroether alcohols,

B is at least one reinforcing monomer copolymerizable with A,

C is a monomer having the general formula X(Y)ₙSi(R)₃₋ₘZₘ wherein

X is a vinyl group copolymerizable with the A and B monomers,

Y is a divalent linking group which is alkylene, arylene, alkarylene, and aralkylene of 1 to 30 carbon atoms which may incorporate ester, amide, urethane, or urea groups,

n is zero or 1;

m is an integer of from 1 to 3,

R is hydrogen, C₁₋₄ alkyl, aryl, or alkoxy,

Z is a monovalent siloxane polymeric moiety; and

D is at least one free radically polymerizable acrylate or methacrylate copolymer.

Such polymers and their manufacture are disclosed in U.S. Pat. Nos. 5,209,924 and 4,972,037, which are hereby incorporated by reference in their entirety.

More specifically, the preferred polymer is a combination of A, C, and D monomers wherein A is a polymerizable acrylic or methacrylic ester of a fluoroalkylsulfonamido alcohol, and where D is a methacrylic acid ester of a C₁₋₂ straight or branched chain alcohol, and C is as defined above. Most preferred is a polymer having moieties of the general formula:

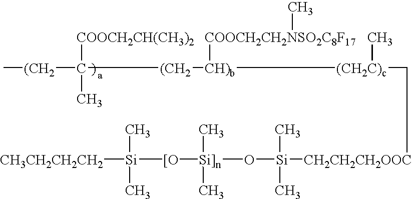

wherein each of a, b, and c has a value in the range of 1-100,000, and the terminal groups are selected from the group consisting of a C₁₋₂₀ straight or branched chain alkyl, aryl, and alkoxy and the like. These polymers may be purchased from Minnesota Mining and Manufacturing Company under the tradenames “Silicone Plus” polymers. Most preferred is poly(isobutyl methacrylate-co-methyl FOSEA)-g-poly(dimethylsiloxane) which is sold under the tradename SA 70-5 IBMMF.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:

A is at least on free radically polymerizable vinyl, methacrylate, or acrylate monomer;

B, when present, is at least one reinforcing monomer copolymerizable with A,

C is a monomer having the general formula:
X(Y)ₙSi(R)₃₋ₘZₘ
wherein:

X is a vinyl group copolymerizable with the A and B monomers;

Y is a divalent linking group;

n is zero or 1;

m is an integer of from 1 to 3;

R is hydrogen, C₁₋₁₀ alkyl, substituted or unsubstituted phenyl, C₁₋₁₀ alkoxy; and

Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, or ionic group (such as quaternary ammonium, carboxylate salt, sulfonic acid salt, and so on).

The C monomer is as above defined.

Examples of other suitable copolymers that may be used herein, and their method of manufacture, are described in detail in U.S. Pat. No. 4,693,935, Mazurek, U.S. Pat. No. 4,728,571, and Clemens et al., both of which are incorporated herein by reference. Additional grafted polymers are also disclosed in EPO Application 90307528.1, published as EPO Application 0 408 311, U.S. Pat. No. 5,061,481, Suzuki et al., U.S. Pat. No. 5,106,609, Bolich et al., U.S. Pat. No. 5,100,658, Bolich et al., U.S. Pat. No. 5,100,657, Ansher-Jackson, et al., U.S. Pat. No. 5,104,646, Bolich et al., U.S. Pat. No. 5,618,524, issued Apr. 8, 1997, all of which are incorporated by reference herein in their entirety.

(b). Polymers from Ethylenically Unsaturated Monomers

Also suitable for use as film forming polymers are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

Preferred are polymers containing one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include those of the formula: 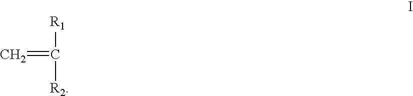
wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200.

More specific types of polymers are where the monofunctional ethylenically unsaturated monomer is of Formula I, above, wherein R₁ is H or a C₁₋₃₀ alkyl, and R₂ is COOM wherein M is a C₁₋₃₀ straight or branched chain alkyl which may be substituted with one or more hydroxy groups.

Even more specific types of polymers are those wherein R₁ is H or CH₃, and R₂ is COOM wherein M is a C₁₋₁₀ straight or branched chain alkyl which may be substituted with one or more hydroxy groups. In the preferred embodiment of the invention, the monofunctional ethylenically unsaturated monomer is a mixture of monomers of Formula I where in one monomer R₁ is H or CH₃ and R₂ is COOM where M is a C₁₋₁₀ alkyl, and where in the second monomer R₁ is H or CH₃, and R₂ is COOM where M is a C₁₋₁₀ alkyl substituted with one or more hydroxy groups.

Di-, tri- and polyfunctional monomers, as well as oligomers, of the above monofunctional monomers may also be used to form the polymer. Suitable difunctional monomers include those having the general formula: 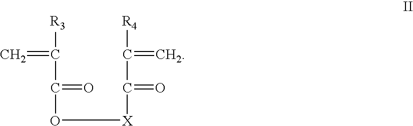
wherein R₃ and R₄ are each independently H, a C₁₋₃₀ straight or branched chain alkyl, aryl, or aralkyl; and X is [(CH₂)ₙO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₙO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas: 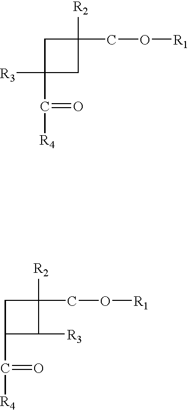
wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane/acrylates copolymer and the polymer is solubilized in isododecane. The monomers mentioned herein can be polymerized with various types of organic groups such as propylene glycol, isocyanates, amides, etc.

One type of organic group that can be polymerized with the above monomers includes a urethane monomer. Urethanes are generally formed by the reaction of polyhydroxy compounds with diisocyanates, as follows: 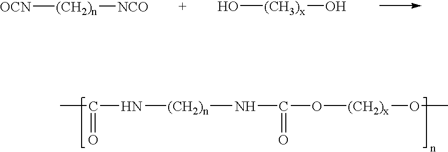
wherein x is 1-1000.

Another type of monomer that may be polymerized with the above comprise amide groups, preferably having the following formula: 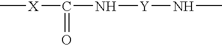
wherein X and Y are each independently linear or branched alkylene having 1-40 carbon atoms, which may be substituted with one or more amide, hydrogen, alkyl, aryl, or halogen substituents.

Another type of organic monomer may be alpha or beta pinenes, or terpenes, abietic acid, and the like.

(c). Silicone Polymers

Also suitable are various types of high molecular weight silicone polymers including those having the formula set forth below, which are commonly known as silicone resins: 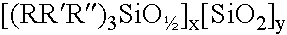
wherein R, R′ and R″ are each independently a C₁₋₁₀ straight or branched chain alkyl or phenyl, and x and y are such that the ratio of (RR′R″)₃SiO_{1/2} units to SiO₂ units ranges from about 0.1 to 1.5 to 1.5 to 0.1.

In one type of silicone resin, R, R′ and R″ are a C₁₋₆ alkyl, and more preferably are methyl and x and y are such that the ratio of (CH₃)₃SiO_{1/2} units to SiO₂ units is about 0.75 to 1; and wherein the trimethylsiloxy silicate contains from about 2.4 to 2.9 weight percent hydroxyl groups. This type of trimethylsiloxysilicate is formed by the reaction of the sodium salt of silicic acid, chlorotrimethylsilane, and isopropyl alcohol. The manufacture of trimethylsiloxy silicate is set forth in U.S. Pat. Nos. 2,676,182; 3,541,205; and 3,836,437, all of which are hereby incorporated by reference. One type of trimethylsiloxysilicate suitable for use is available from Dow Corning Corporation under the tradename 749 Fluid (formerly known as 2-0749), which is a blend of about 40-60% volatile silicone and 40-60% trimethylsiloxysilicate. Dow Corning 749 Fluid in particular, is a fluid containing about 50% trimethylsiloxysilicate and about 50% cyclomethicone. The fluid has a viscosity of 200-700 centipoise at 25° C., a specific gravity of 1.00 to 1.10 at 25° C., and a refractive index of 1.40-1.41. A similar trimethylsiloxysilicate is available from GE Silicones under the tradename SR1000 and is a fine particulate solid material. Yet another type of particulate silicone resin (trimethylsiloxysilicate) is commercially available from Wacker-Chemie under the tradename Belsil TMS 803.

Another type of resinous silicone polymer suitable for use in the invention comprises the silicone esters set forth in U.S. Pat. No. 5,725,845, which is hereby incorporated by reference in its entirety. Other polymers that can enhance adhesion to skin include silicone esters comprising units of the general formula RₐR^{E}_{b}SiO_{[4-(a+b)/2]} or R¹³ₓR^{E}_{y}SiO_{1/2} wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein RE is a carboxylic ester containing radical. More specifically, preferred R_{E} radicals are those wherein the ester group is formed of one or more fatty acid moieties (e.g. of about 2, often about 3 to 10 carbon atoms) and one or more aliphatic alcohol moieties (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety.

Preferably the silicone ester will have a melting point of no higher than about 120° C. It can be a liquid or solid at room temperature. Preferably it will have a waxy feel and a molecular weight of no more than about 100,000 daltons.

Silicone esters having the above formula are disclosed in U.S. Pat. No. 4,725,658 and U.S. Pat. No. 5,334,737, which are hereby incorporated by reference. Specific types of silicone esters include liquid siloxy silicates disclosed in U.S. Pat. No. 5,334,737, e.g. diisostearoyl trimethylolpropane siloxysilicate (prepared in Examples 9 and 14 of this patent), and dilauroyl trimethylolpropane siloxy silicate (prepared in Example 5 of the patent), which are commercially available from General Electric under the tradenames SF 1318 and SF 1312, respectively.

(d). Natural Polymers

Also suitable for use are one or more naturally occuring polymeric materials such as resinous plant extracts including such as rosin, shellac, and the like.

It is further noted that if such other film forming polymers are present, in one preferred embodiment of the invention, the ratio of film forming polymer to very high viscosity silicone gum ranges from about 0.1 to 20 parts polymer to 1 part silicone gum, more preferably from about 0.5 to 3 parts polymer to about 1 part silicone gum.

6. Water or Non-Aqueous Solvents

The composition of the invention may be in the anhydrous or emulsion form. If the latter, the composition may comprise from about 0.1-95%, preferably from about 0.5-80%, more preferably 1-75% by weight of the total composition of water or other polar solvents such as butylene glycol, propylene glycol, ethanol, isopropanol, or mixtures thereof.

7. Surfactants

One or more surfactants may be present, whether the composition is in the anhydrous or emulsion form. If present, the surfactant may range from about 0.001-40%, preferably about 0.1-15%, more preferably about 0.5-10% by weight of the total composition. The surfactant may be in the nonionic, cationic, anionic, zwitterionic, or amphoteric form. Preferably, if surfactants are present they are nonionic.

(a). Nonionic Organic Surfactants

Suitable nonionic surfactants include alkoxylated alcohols, or ethers, formed by the reaction of an alcohol with an alkylene oxide, usually ethylene or propylene oxide. Preferably the alcohol is a fatty alcohol having 6 to 30 carbon atoms. Examples of such ingredients include Beheneth 5-30, which is formed by the reaction of behenyl alcohol and ethylene oxide where the number of repeated ethylene oxide units ranges from 5 to 30; Ceteareth 2-100, formed by the reaction of a mixture of cetyl and stearyl alcohol with ethylene oxide, where the number of repeating ethylene oxide units in the molecule ranges from 2 to 100; Ceteth 1-45 which is formed by the reaction of cetyl alcohol and ethylene oxide, and the number of repeating ethylene oxide units ranges from 1 to 45, and so on. Other alkoxylated alcohols are formed by the reaction of fatty acids, mono-, di- or polyhydric alcohols, and alkylene oxides. For example, compounds formed by the reaction of C₆₋₃₀ fatty carboxylic acids, polyhydric alcohols (such as monosaccharides such as glucose, galactose, glycerin, methyl glucose) and an alkoxylated alcohol (such as steareth, beheneth, ceteareth, and the like) are also suitable.

Also suitable as the nonionic surfactant are alkyoxylated carboxylic acids, which are formed by the reaction of a carboxylic acid with an alkylene oxide or with a polymeric ether. The resulting products have the general formula: 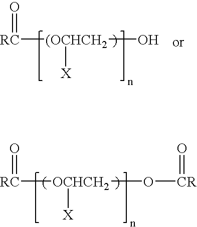
where R is a C₁₋₃₀ straight or branched chain saturated or unsaturated alkyl, X is hydrogen or lower alkyl, and n is the number of polymerized alkoxy groups, which may range from 2 to 100,000. In the case of the diesters, the two RCO-groups do not need to be identical.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula: 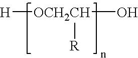
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

(b). Silicone Surfactants

Suitable silicone surfactants that may be used in the compositions of the invention include those that have a polymeric backbone having siloxy units that have linear repeating units, e.g. di(lower)alkylsiloxy units, preferably dimethylsiloxy units. The silicone surfactant has a hydrophilic portion, which is generally achieved by substitution onto the polymeric backbone of a radical that confers hydrophilic properties to a portion of the molecule. The hydrophilic radical may be substituted on a terminus of the silicone, or on any one or more repeating units of the polymer. In general, the repeating dimethylsiloxy units of modified polydimethylsiloxane surfactants are lipophilic in nature due to the methyl groups, which confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the silicone surfactant, if used in the compositions of the invention, should have at least one hydrophilic portion and one lipophilic portion.

The term “hydrophilic radical” means a radical that, when substituted onto the silicone polymer backbone, confers hydrophilic properties to the substituted portion of the polymer. Examples of radicals that will confer hydrophilicity are hydroxy-polyethyleneoxy, hydroxyl, carboxylates, sulfonates, sulfates, phosphates, or amines.

The term “lipophilic radical” means an organic radical that, when substituted onto the silicone polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. The C₁₋₄₀ alkyl may be non-interrupted, or interruped by one or more oxygen atoms, a benzene ring, amides, esters, or other functional groups.

The silicone surfactants may include those having the following general formulas:

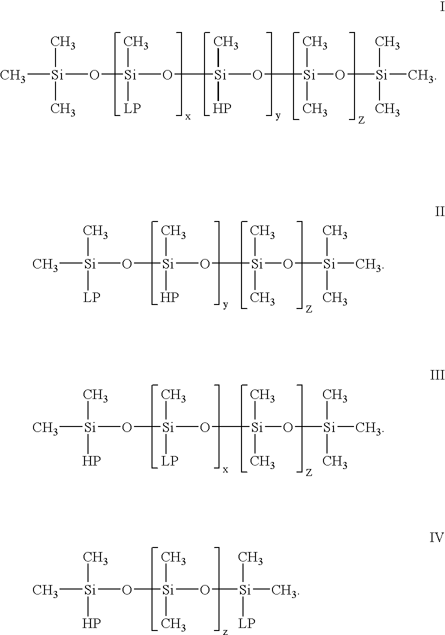

wherein LP is a lipophilic radical

HP is a hydrophilic radical
x is 0-5000
y is 0-5000, and
z is 0-5000, with the proviso that the organosiloxane contains at least on hydrophilic radical and at least one lipophilic radical.

More preferred are compounds of the generic formula I, above, wherein LP is a lipophilic radical which is a C₁₋₄₀ straight or branched chain alkyl, HP is a hydrophilic radical containing hydroxy-polyethyleneoxy. Most preferred is a compound of the formula: 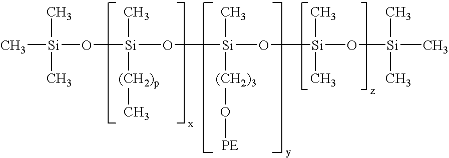
wherein p is 10-40, preferably 12-20, most preferably 15, a is 1-50,000, b is 1-50,000, and
PE is (—C₂H₄O)ₐ(—C₃H₆O)_{b}—H
where x, y, z, a, and b are such that the maximum molecular weight of the polymer is approximately 50,000. Silicone surfactants useful in the compositions of the invention are commercially available from Goldschmidt Corporation under the ABIL tradename. One type of such surfactant is cetyl dimethicone copolyol and has the tradename ABIL WE 09 or ABIL WS 08. The cetyl dimethicone copolyol may be used alone or in conjunction with other non-silicone organic surfactants. For example, the cetyl dimethicone copolyol may be in a mixture with other non-silicone organic surfactants and emollients. In particular, blends of 25-50% of the organosiloxane surfactant, 25-50% of a non-silicone organic surfactant, and 25-50% by weight emollients or oils are preferred. For example, the mixtures identified by the C.T.F.A. names cetyl dimethicone copolyol (and) polyglyceryl 4-isostearate (and) hexyl laurate, or cetyl dimethicone copolyol (and) polyglyceryl-3 oleate (and) hexyl laurate both work well. These blends contain approximately 25-50% of each ingredient, for example ABIL WE 09 contains approximately, by weight of the total ABIL composition, 25-50% cetyl dimethicone copolyol, 25-50%, polyglyceryl 4-isostearate, and 25-50% of hexyl laurate which is an emollient or oil.

Another type of silicone surfactant suitable for use in the compositions of the invention are sold by Union Carbide under the Silwet™ trademark. These surfactants are represented by the following generic formulas:
(Me₃Si)_{y-2}[(OSiMe₂)_{x/y}O—PE]_{y}
wherein

PE=-(EO)ₘ(PO)ₙR

R=lower alkyl or hydrogen

Me=methyl

EO is polyethyleneoxy

PO is polypropyleneoxy

m and n are each independently 1-5000

x and y are each independently 0-5000, and 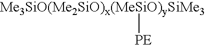
wherein

PE=—CH₂CH₂CH₂O(EO)ₘ(PO)ₙZ

Z=lower alkyl or hydrogen, and

Me, m, n, x, y, EO and PO are as described above,

with the proviso that the molecule contains a lipophilic portion and a hydrophilic portion. Again, the lipophilic portion can be supplied by a sufficient number of methyl groups on the polymer backbone.

One particular type of silicone surfactant is sold under the Silwet™ brand and has the following general formula: 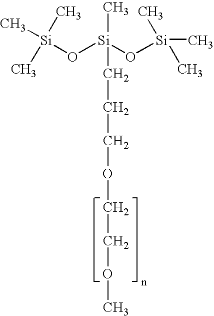
wherein n is 1-10, preferably 8.

Generally silicone co-emsulfiers suitable for use in the compositions of the invention are known by the INCI name dimethicone copolyol and cetyl dimethicone copolyol.

Examples of other silicone surfactants include amino/polyoxyalkyleneated polydiorganosiloxanes disclosed in U.S. Pat. No. 5,147,578. Also suitable are organosiloxanes sold by Goldschmidt under the ABIL trademark including ABIL B-9806, as well as those sold by Rhone-Poulenc under the Alkasil tradename. Also, organosiloxane surfactants sold by Amerchol under the Amersil tradename, including Amersil ME-358, Amersil DMC-287 and Amersil DMC-357 are suitable. Dow Corning surfactants such as Dow Corning 3225C Formulation Aid, Dow Corning 190 Surfactant, Dow Corning 193 Surfactant, Dow Corning Q2-5200, and the like are also suitable

Additional suitable cationic, anionic, zwitterionic, and amphoteric surfactants are further described in U.S. Pat. No. 5,534,265, which is hereby incorporated by reference in its entirety.

8. Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

9. Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, famesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2, 2000.

The invention will be further described in connection with the following examples which are set forth for the purposes of illustration only.

### EXAMPLE 1

A transfer resistant lipstick was made as follows:

| Ingredient | % by weight |
|---|---|
| Mixture of 25 parts dimethicone gum 4-and 75 parts | 40.00 |
| 1 centistoke dimethicone* | |
| Polyethylene | 10.00 |
| Dimethicone, 1 centistoke | 33.00 |
| Octyl isononanoate | 5.00 |
| 50 parts pigment/50 parts 1 centistoke dimethicone | 12.00 |

*viscosity of mixture was 10,160 centistokes at 20° C. as measured by Brookfield LVT viscometer, using T-D spindle at 5 RPM for 1 minute.

The composition was prepared by grinding the pigments in 1 centistoke dimethicone. The wax was melted and the remaining ingredients were added and mixed well. The lipstick composition was poured into vials. When applied to the lips, the lipstick had excellent application, provided a matte finish on the lips, and wore about eight hours.

### EXAMPLE 2

Transfer resistant lipsticks were prepared as follows:

| Ingredient | 1 | 2 | 3 | 4 | 5 | 6 |
|---|---|---|---|---|---|---|
| Trimethylsiloxysilicate | 22.00 | 22.00 | 22.00 | 22.00 | 21.00 | 21.00 |
| 1000 centistoke | 16.00 | 8.00 | 10.00 | 10.00 | 12.00 | 13.00 |
| dimethicone | | | | | | |
| Isododecane | 9.00 | 17.00 | 17.00 | 9.00 | 8.00 | 10.00 |
| Mixture of 50% | 13.00 | 13.00 | 13.00 | 13.00 | 13.00 | 13.00 |
| pigment/50% | | | | | | |
| isododecane | | | | | | |
| Mixture of 25 parts | 40.00 | 8.00 | 6.00 | 6.00 | 6.00 | 3.00 |
| dimethicone gum 75 | | | | | | |
| parts isododecane | | | | | | |
| 100,000 centistoke | — | 8.00 | 6.00 | 6.00 | 6.00 | 3.00 |
| dimethicone | | | | | | |

*viscosity of mixture 8,350 centistokes at 20° C. as measured by Brookfield LVT viscometer, using T-D spindle at 5 RPM for 1 minute.

The compositions were prepared by grinding the pigments in isododecane. The remaining ingredients were combined with the pigment grind, mixed well, and poured into vials. The lipsticks were tested by applying to the lips, allowing to dry, and rating gloss, transfer resistance, wear, application, and tack. The results were as follows:

1: Good gloss, some transfer. Stayed on after eating two meals. Ratio of silicone resin (trimethylsiloxysilicate) to dimethicones+esters (also referred to as ε Mod)=0.85. Composition had 48% solids.

2: Good gloss, some transfer. The finish was very slightly tacky on the lips. The ε Mod was about 0.92. Composition had 46% solids.

3: Good gloss, very slight transfer. The ε Mod was about 0.92. Composition had 46% solids.

4: Good gloss, slight transfer. Composition was slightly tacky on the lips. The ε Mod was about 0.85. Composition had 48% solids.

5: Good gloss, transferred. Composition less tacky than (4), above. The ε Mod was about 0.75.

6: Good gloss, transferred. Slightly tacky.

### EXAMPLE 3

Lipstick compositions were prepared as follows:

| Ingredient | 1 | 2 | 3 | 4 |
|---|---|---|---|---|
| Trimethylsiloxysilicate | 20.00 | 20.00 | 18.40 | 18.40 |
| Mixture of 25 parts | 52.50 | 52.50 | 46.50 | 46.50 |
| dimethicone gum having | | | | |
| viscosity greater than 25 | | | | |
| million and 75 parts | | | | |
| isododecane | | | | |
| Isododecane | 1.00 | 1.00 | 1.00 | 1.00 |
| Mixture of 50 parts pigment | 13.00 | 13.00 | 13.00 | 13.00 |
| and 50 parts isododecane | | | | |
| Mica | 1.00 | 1.00 | 1.00 | 1.00 |
| Dimethicone, 350 centistokes | 3.00 | 3.00 | 3.00 | 3.00 |
| Dibutyl adipate | 5.00 | 5.00 | 4.00 | 4.00 |
| Mixture of 10 parts | 5.00 | 5.00 | 5.00 | 5.00 |
| Quaternium-18 hectorite and | | | | |
| 90 parts isododecane | | | | |
| Methyl paraben | — | 0.30 | 0.30 | 0.30 |
| Propyl paraben | — | 0.10 | 0.10 | 0.10 |
| BHT | — | 0.10 | 0.10 | 0.10 |
| Polyethylene | — | 1.00 | 1.00 | — |

The compositions were prepared by grinding the pigments in isododecane. The remaining ingredients were mixed well and added to the pigment grind. The composition was poured into vials. The compositions were further tested as noted below:

1: The viscosity was 38,000 centistokes at 25° C.

2: The viscosity was 26,210 centistokes at 25° C. The composition was applied to the lips and stayed on the lips for nine hours.

3: The viscosity was 31,800 centistokes at 25° C.

4: The viscosity was 6,375 centistokes at 25° C.

### EXAMPLE 4

Lipstick compositions were prepared as follows:

| Ing. | 1 | 2 | 3 | 4 | 5 | 6 | 7 | 8 | 9 |
|---|---|---|---|---|---|---|---|---|---|
| Trimethylsiloxysilicate | 18.40 | 18.40 | 18.40 | 18.40 | 30.00 | 30.00 | 28.00 | 26.00 | 25.00 |
| Isododecane | 6.00 | 6.00 | 6.00 | 6.00 | 6.00 | 6.00 | 6.00 | 5.00 | 3.00 |
| * | 45.00 | 45.00 | 45.00 | 45.00 | 30.00 | 30.00 | 33.00 | 36.00 | 39.00 |
| Pigment | 13.00 | 13.00 | 13.00 | 13.00 | 13.00 | 13.00 | 13.00 | 13.00 | 13.00 |
| Grind** | | | | | | | | | |
| Mica | 1.00 | 1.00 | 1.00 | 1.00 | 1.00 | 1.00 | 1.00 | 1.00 | 1.00 |
| *** | 10.00 | — | — | — | — | — | — | — | — |
| PEG-4 | — | 10.00 | — | — | — | — | — | — | — |
| diheptanoate | | | | | | | | | |
| Octyl dodecyl | — | — | 10.00 | — | 15.00 | 12.00 | 11.00 | 11.00 | 10.00 |
| neopentanoate | | | | | | | | | |
| Glyceryl | — | — | — | 10.00 | — | — | — | — | — |
| trioctanoate | | | | | | | | | |
| **** | — | — | — | — | — | 8.00 | 8.00 | 8.00 | 8.00 |

* 25 parts dimethicone gum having viscosity of greater than 25 million and 75 parts isododecane.
**50 parts pigment and 50 parts isododecane.
*** Diglyceryl diisostearate.
**** 10 parts Quaternium-18 hectorite and 90 parts isododecane.

The compositions were prepared by grinding the pigment in isododecane. The remaining ngredients were combined and mixed well to form the final composition. The compositions were poured into vials. The compositions were further tested and evaluated as follows:

1: Composition not internally compatible.

2: Moderate gloss when applied to hand, but did not adhere to skin.

3: When applied to lips showed slight to moderate gloss and very slight transfer.

4: When applied to lips showed slight to moderate gloss and very slight transfer.

5: When applied to lips shows good gloss, slight transfer, and good application.

6: When applied to lips showed good gloss. Wore 5 hours on the lips but did not wear through a meal.

7: When applied to hand showed good gloss and no transfer.

8: When applied to hand showed good gloss and no transfer.

9: Then applied to hand showed moderate gloss and no transfer.

### EXAMPLE 5

A mascara formula was made as follows:

| Ingredient | % by weight |
|---|---|
| Water | QS |
| Acacia Senegal gum | 1.75 |
| Triethanolamine | 2.25 |
| Lecithin, polysorbate 20, sorbitan laurate, | 0.20 |
| propylene glycol stearate, propylene glycol laurate | |
| Simethicone | 0.20 |
| Hydroxyethylcellullose | 0.20 |
| Panthenol | 0.10 |
| Nylon-12 | 1.50 |
| Methyl paraben | 0.35 |
| Polyethylene | 0.80 |
| Iron oxides | 9.00 |
| Stearic acid | 5.60 |
| Paraffin | 10.80 |
| Beeswax | 2.80 |
| Phenoxyethanol | 1.00 |
| Propyl paraben | 0.15 |
| Hydrogenated stearyl olive ester | 1.00 |
| Carnauba wax | 3.50 |
| Tocopheryl acetate | 0.10 |
| Trimethylsiloxysilicate | 2.00 |
| Cyclomethicone, dimethiconol | 2.70 |
| Cyclomethicone, hydrogenated polyisobutene, 3 | 0.65 |
| dimethicone crosspolymer | |
| Phytantriol | 0.40 |
| Hydrolyzed silk | 0.05 |
| Retinyl palmitate | 0.01 |

The composition was prepared by separately combining the oil phase and water phase ingredients. The separate phases were then combined and mixed well to emulsify.

### EXAMPLE 6

Blush and eyeshadow formulas were made as follows:

| | % by weight | |
|---|---|---|
| Ingredient | Blush | Eyeshadow |
| Ethylhexyl palmitate | — | 21.43 |
| Dimethicone | 16.40 | 18.61 |
| Isotridecyl isononanoate | 12.02 | — |
| Hydroxylated lanolin | — | 1.90 |
| Sorbitan trioleate | — | 0.50 |
| Neopentyl glycol diethylhexanoate | 14.20 | — |
| Silicone gum, Dow Corning (R) 4-7034 INT | 3.65 | 3.50 |
| Cyclomethicone, trimethylsiloxysilicate | — | 4.75 |
| Phenyl trimethicone | 2.00 | 0.09 |
| Tribehenin | — | 9.75 |
| Candelilla wax | — | 0.10 |
| Silica silylate | — | 1.25 |
| Sorbitan trioleate | 0.50 | — |
| BHT | 0.10 | 0.05 |
| Sorbic acid | 0.20 | — |
| Polyethylene | 5.50 | — |
| Phenyl trimethicone, distearalkonium hectorite, | 5.00 | — |
| triethyl citrate | | |
| Mica, bismuth oxychloride, | 1.00 | — |
| calcium aluminum borosilicate | | |
| Mica, barium sulfate, titanium dioxide | 0.50 | — |
| Talc, ethylene/methacrylate copolymer, | 5.00 | — |
| isopropyl titanium triisostearate | | |
| Talc | — | 4.77 |
| Boron nitride | 4.00 | — |
| Bismuth oxychloride, silica, mica | 0.50 | — |
| Mica | 0.75 | — |
| Talc, lecithin | 4.35 | — |
| Titanium dioxide, polyethylene | 1.00 | — |
| Cyclomethicone, dimethicone, | 2.76 | — |
| trimethylsiloxysilicate, iron oxides | | |
| Cyclomethicone, dimethicone, | — | 0.10 |
| trimethylsiloxysilicate, titanium dioxide | | |
| Magnesium carbonate | | 1.90 |
| Red 6 Lake/isotridecyl isononanoate | 0.10 | — |
| Methyl methacrylate crosspolymer | 4.00 | — |
| Nylon-12 | 2.50 | 0.48 |
| Polymethylmethacrylate | — | 0.47 |
| Silica | 2.50 | — |
| Acrylates copolymer | 0.05 | — |
| Dimethicone, cyclomethicone | 0.35 | — |
| Alumina | 1.00 | — |
| HDI/trimethylol hexyllactone crosspolymer, silica | 3.00 | — |
| Mica, titanium dioxide | 5.25 | 20.00 |
| Mica | — | 8.00 |
| Bismuth oxychloride, silica, mica | — | 0.95 |
| Talc, lecithin | 0.10 | — |
| Isotridecyl isononanoate | 0.10 | — |
| Phenoxyethanol | 1.00 | — |
| Parabens | 0.60 | 1.40 |

The compositions were prepared by grinding the pigments in a portion of the oils. The waxes and oils were combined and heated. The pigment grind was added to the molten waxes and oils along with the other ingredients. The mixture was poured into pans and cooled.

### EXAMPLE 7

A foundation makeup composition was prepared as follows:

| Ingredient | % by weight |
|---|---|
| Cyclomethicone, trimethylsiloxysilicate | 34.25 |
| Cyclomethicone | 1.66 |
| Cetyl PEG/PPG-10/1 dimethicone | 0.25 |
| Dimethicone | 2.65 |
| Bisabolol | 0.05 |
| Tribehenin | 1.50 |
| Silicone gum (Dow Corning (R) 4-7034 INT) | 4.50 |
| Sorbitan sesquioleate | 0.13 |
| Titanium dioxide/methicone | 8.00 |
| Silk powder | 0.10 |
| Mica, methicone | 1.30 |
| Iron oxides, methicone/boron nitride (50:50) | 0.80 |
| Iron oxides, methicone | 3.45 |
| Nylon-12 | 5.30 |
| Boron nitride | 9.54 |
| Water | QS |
| Sodium chloride | 1.00 |
| Tetrasodium EDTA | 0.01 |
| Butylene glycol | 4.50 |
| Parabens | 0.30 |
| SD alcohol 40B | 3.25 |
| Ethylene brassylate | 0.10 |
| Tocopheryl acetate | 0.05 |

The composition was prepared by grinding the pigments and powders in a portion of the cyclomethicone/trimethylsiloxysilcate and dimethicone. The remaining cyclomethicone/trimethylsiloxysilicate mixture, silicone gum, cyclomethicone, and dimethicone were added and mixed well. The water and water phase ingredients were separately mixed and then combined with the oily phase ingredients and mixed well to form an emulsion.

### EXAMPLE 8

An eyeshadow formula was made as follows:

| Ingredient | % by weight |
|---|---|
| Acrylates copolymer | 0.30 |
| Zinc stearate | 1.50 |
| Polypropylene | 3.20 |
| Bismuth oxychloride, cyclomethicone, acrylates | 5.00 |
| dimethicone copolymer, trimethylsiloxysilicate | |
| Boron nitride, cyclomethicone, acrylates | 15.00 |
| dimethicone copolymer, trimethylsiloxysilicate | |
| Talc, cyclomethicone, acrylates dimethicone | 5.00 |
| copolymer, trimethylsiloxysilicate | |
| Parabens | 0.50 |
| Iron oxides | 4.25 |
| Mica | 48.24 |
| Mica, mineral oil, methicone | 6.00 |
| Nylon-12 | 2.00 |
| Butylene glycol dicaprylate/dicaprate | 1.00 |
| Cetyl dimethicone | 1.00 |
| Phenoxyethanol | 1.00 |
| Dimethicone | 5.00 |
| Dimethicone gum (Dow Corning (R) 4-7034 INT) | 1.00 |
| Mica | 0.01 |

The composition was prepared by grinding the pigments and powders in a portion of the oils. The waxes and oils were combined and heated. The pigment/powder grind was mixed with the waxes and oils and the composition was poured into containers and allowed to cool.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A color cosmetic composition comprising at least one very high viscosity silicone gum in a cosmetically acceptable carrier, wherein the high viscosity silicone gum is not dimethiconol.

2. The composition of claim 1 wherein the very high viscosity silicone gum has a viscosity ranging from about 12 million to 900 million centistokes at 25° C.

3. The composition of claim 2 wherein the silicone gum has a viscosity ranging from about 15 million to 750 million centistokes at 25° C.

4. The composition of claim 2 wherein the silicone gum has a viscosity ranging from about 20 million to 600 million centistokes at 25° C.

5. The composition of claim 2 wherein the silicone gum has a viscosity ranging from about 20 million to 500 million centistokes at 25° C.

6. The composition of claim 1 wherein the silicone gum has the general formula: 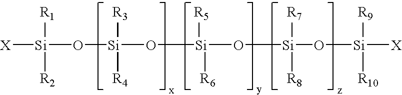
wherein:
R₁ through R₉ are each independently an alkyl having 1 to 30 carbon atoms, aryl, or aralkyl; and X is a C₁₋₃₀ alkyl, hydroxyl, or vinyl; and wherein x, y, or z may be zero provided no more than two of x, y, or z are zero at any one time, and further that x, y, and z are such that the silicone gum has a viscosity of about 12 million centistokes, ranging up to about 900 million centistokes at 25° C.

7. The composition of claim 1 further comprising at least one volatile solvent.

8. The composition of claim 7 wherein the volatile solvent is a silicone, paraffinic hydrocarbon, or mixtures thereof.

9. The composition of claim 8 wherein the volatile solvent is a linear or cyclic silicone.

10. The composition of claim 9 wherein the linear silicone is selected from the group consisting of hexamethyldisiloxane, octamethyltrisiloxane, decamethyltetrasiloxane, dodecamethylpentasiloxane, and mixtures thereof.

11. The composition of claim 9 wherein the cyclic silicone is selected from the group consisting of decamethylcyclopentasiloxane, dodecamethylcyclohexasiloxane, and mixtures thereof.

12. The composition of claim 8 wherein the volatile solvent is a paraffinic hydrocarbon.

13. The composition of claim 12 wherein the volatile solvent is a C₆₋₁₆ paraffinic hydrocarbon.

14. The composition of claim 13 wherein the volatile solvent is selected from the group consisting of C₉₋₁₁ isoparaffins, isododecane, isohexadecane, and mixtures thereof.

15. The composition of claim 1 further comprising at least one structuring agent.

16. The composition of claim 15 wherein the structuring agent is a wax.

17. The composition of claim 16 wherein the structuring agent is present ranging from about 0.1-75% by weight of the total composition.

18. The composition of claim 17 wherein the structuring agent is a natural, synthetic, mineral, or silicone wax.

19. The composition of claim 18 wherein the structuring agent is a synthetic wax.

20. The composition of claim 19 wherein the structuring agent comprises polyethylene.

21. The composition of claim 1 further comprising an ester.

22. The composition of claim 21 wherein the ester is a mono-, di-, or triester.

23. The composition of claim 22 wherein the monoester is an ester of a C₁₋₃₀ straight or branched chain alcohol and a C₁₋₃₀ straight or branched chain carboxylic acid.

24. The composition of claim 23 wherein the monoester is an ester of C₁₋₁₀ straight or branched chain alcohol and a C₁₋₁₀ straight or branched chain carboxylic acid.

25. The composition of claim 1 further comprising a triester.

26. The composition of claim 25 wherein the triester is the reaction product of a tricarboxylic acid and a C₁₋₃₀ straight or branched chain alcohol, or the reaction product of a C₁₋₃₀ straight or branched chain alcohol having at least three hydroxyl groups and C₁₋₃₀ straight or branched chain fatty acids.

27. The composition of claim 26 wherein the triester is the reaction product of citric acid and trioctyldodecyl alcohol.

28. The composition of claim 26 wherein the triester is the reaction product of a C₁₋₃₀ straight or branched chain alcohol having at least three hydroxyl groups and a C₁₋₃₀ carboxylic acid, or a C₁₋₃₀ straight of branched chain alcohol and a C₁₋₃₀ tricarboxylic acid.

29. The composition of claim 28 wherein the triester is the reaction product of glycerin and octanoic acid and the triester is glyceryl trioctanoate.

30. The composition of claim 1 further comprising a diester.

31. The composition of claim 30 wherein the diester is the reaction product of a C₁₋₃₀ straight or branched chain alcohol having at least two hydroxyl groups and a C₁₋₃₀ straight or branched chain carboxylic acid, or the reaction product of a C₁₋₃₀ straight or branched chain alcohol and a C₁₋₃₀ straight or branched chain dicarboxylic acid.

32. The composition of claim 30 wherein the diester is the reaction product of polyethylene glycol and heptanoic acid and the diester is polyethylene glycol diheptanoate.

33. The composition of claim 30 wherein the diester is the reaction product of glycerin and isostearic acid and the ester is diglyceryl diisostearate.

34. The composition of claim 1 further comprising a non-volatile silicone having a viscosity ranging from about 8 to 500,000 centistokes at 25° C.

35. The composition of claim 1 wherein the composition is a mascara, blush, eyeshadow, foundation makeup, or lipstick.

36. The composition of claim 1 further comprising pigments, powders, or mixtures thereof.

37. The composition of claim 36 wherein the pigments are organic pigments, inorganic pigments, or mixtures thereof.

38. The composition of claim 36 wherein the powders are spherical, lamellar, or mixtures thereof.

39. The composition of claim 36 wherein the powders are selected from the group consisting of mica, talc, titanium dioxide, boron nitride, nylon, and mixtures thereof.

40. The composition of claim 39 wherein the pigments and powders combined are present ranging from about 0.1-85% by weight of the total composition.

41. The composition of claim 1 comprising:
at least one silicone gum having a viscosity ranging from about 12 million to about 900 million centistokes at 25° C. wherein the silicone gum is not dimethiconol.
at least one volatile solvent,
at least one non-volatile silicone; and
at least one pigment.

42. The composition of claim 41 wherein the volatile solvent is a linear silicone, cyclic silicone, paraffinic hydrocarbon or mixtures thereof.

43. The composition of claim 42 wherein the linear silicone is selected from the group consisting of hexamethyldisiloxane, octamethyltrisiloxane, decamethyltetrasiloxane, dodecamethylpentasiloxane, and mixtures thereof.

44. The composition of claim 42 wherein the cyclic silicone is selected from the group consisting of decamethylcyclopentasiloxane, dodecamethylcyclohexasiloxane, and mixtures thereof.

45. The composition of claim 42 wherein the volatile solvent is a paraffinic hydrocarbon selected from the group consisting of C₉₋₁₁ isoparaffins, isododecane, isohexadecane, and mixtures thereof.

46. A cosmetic composition comprising a very high viscosity silicone gum solvated in at least one viscosity decreasing, composition compatible solvent.

47. The composition of claim 46 wherein the very high viscosity silicone gum has a viscosity ranging from about 12 million to about 900 million centistokes at 25° C., and the viscosity decreasing, composition compatible solvent comprises a volatile paraffinic hydrocarbon.

48. The composition of claim 46 wherein the very high viscosity silicone gum has a viscosity ranging from about 12 million to about 900 million centistokes at 25° C., and the viscosity decreasing, composition compatible solvent comprises a linear volatile silicone.

49. The composition of claim 46 wherein the very high viscosity silicone gum has a viscosity ranging from about 12 million to about 900 million centistokes at 25° C., and the viscosity decreasing, composition compatible solvent comprises a cyclic volatile silicone and wherein the composition is substantially free of crystalline structuring agents.

50. The composition of claim 48 wherein the linear volatile silicone is selected from the group consisting of hexamethyldisiloxane, octamethyltrisiloxane, decamethyltetrasiloxane, dodecamethylpentasiloxane, and mixtures thereof.

51. A color cosmetic composition comprising at least one very high viscosity dimethiconol gum in combination with a linear volatile silicone.
